(19) United States
(12) Patent Application Publication
(10) Pub. No.: US 2006/0134597 A1
(43) Pub. Date: Jun. 22, 2006

(54) Apparatus and method for rapid separation of cells without using density gradient and antibodies

(75) Inventor: Jia-Ming Chang, Taipei (TW)

Correspondence Address: HDSL, 4331 STEVENS BATTLE LANE, FAIRFAX, VA, 22033, US

(73) Assignee: Chih Shin biomedical Technology Co., Ltd.

(21) Appl. No.: 11/119,908

(22) Filed: May 3, 2005

(30) Foreign Application Priority Data
Dec. 16, 2004 (TW) 093139057

(51) Int. Cl. A01N 1/02; C12M 1/14

(52) U.S. Cl. 435/2; 435/308.1; 435/299.1

(57) ABSTRACT

A method and an apparatus for rapid cell separation without using density gradient and antibody but using a column packed with resin particles. The interactions between cell surfaces and resin particles resulting in the different retention time of different cells in the column contribute to the separation of the specific cells from the mixed cell population. Therefore, no antibodies and specific chemical reagents are used in cell separation maintain the physiological status of separated cells. This invention can also apply to clinical use for fast and massive separation of blood sub-population, for the remove of leukemia cells from normal leucocytes in vivo or in vitro, and particularly for drug screening through the interaction between drugs and specific and non-specific cells.

### BACKGROUND OF THE INVENTION

1. Field of the Invention

The invention relates to an apparatus and a method for rapid separation of cells without using density gradient and antibody, and in particular, to an apparatus and a method for rapid separation of cells based on the different interactions between various cells with respect to the resin packed in a column due to difference of physical properties between molecules on surfaces of the various cells so that each kind of different cells has its own retention time in the column different to the retention time of other cells, thereby different kind of cells can be separated correspondingly.

2. Description of the Related Art

In current pharmaceutical studies, researchers wish to find the drug target site on cells so as to understand the action mechanism of drug on cells in order to develop more efficient or safer drugs. In the experimental design, homogeneous cells are used to demonstrate the action mechanism of drugs so as to understand the specific response from a particular cell population to a particular drug, and to identify the effect of a drug on different tissue. Thus, the separation and purification of cells play a highly important role in pharmacological experiments. Providing a unique population of cell means not only to provide a directly observable object, but also to understand the action mechanism of a drug in a particular cell.

There are two kind of conventional methods for separating cells, namely, a method in which a special solution, such as Ficoll cell separation medium or Percoll cell separation medium (Percoll, a particulate silica gel treated with polyvinylpyrolidone (PVP)) was used, and based on the gradient due to centrifugal force or varying proportions of substances added, different kinds of cells can be separated into respective density zones in consistent with their own cell density by virtue of centrifugation. For example, U.S. Pat. No. 6,641,517 disclosed an apparatus capable of producing good gradient. Separation of cells by means of density gradient, however, is a difficult operation and time-consumed. Furthermore, the cell separation medium could be toxic on cells to be separated as well as affect the quality of cells.

Another method for separating cells takes advantage of a specific antibody that can recognize the surface molecule on a cell. This antibody can conjugate covalently or through affinity with a fluorescent substance or substance comprising iron-containing component. In case of using an antibody conjugated with a fluorescent substance, a cell sorter can be used to distinguish a particular fluorescence, and meanwhile, upon rendering cells to be charged, specific cells can be separated under an applied electric field, such as described in U.S. Pat. No. 4,629,687. On the other hand, in case of using antibody conjugated with an iron-containing substance, a magnetic field can be used to separate cells labeled with that antibody. Apparatuses and improved apparatuses developed based on this principle were described in, for example, U.S. Pat. Nos. 5,240,856; 5,684,712; 5,691,208; 5,705,059; 5,711,871 and 6,468,432. However, since these apparatuses screen particular cells using their respective antibody, in addition to the high cost of reagents, the development of antibodies might be a limiting factor and at the same time, the utilization of an antibody may render the cell separated being incapable of being used directly in clinical application.

Alternatively, an approach for separating cells by virtue of surface affinity was proposed by Shibusawa in 1999, in which antibodies bound on the resin comprising a stationary phase or on glass beads were used to separate cells by eluting with a competitive substance in buffer solution. As described in the article published by Shibusawa (Shibusawa (1999) J. Chromatography B 722, 71-88), Sepharose 6B or Chromagel A4 combined with polyethylene glycol (PEG) or polypropylene glycol (PPG) were used as the stationary phase in the separation of cells such as granulocytic leukocyte, monocytic leukocyte or erythrocyte, but not in effective separation of certain sub-populations of mononuclear cells such as, for example, T lymphocyte, B lymphocyte and monocytes.

In view of the forgoing, there are many disadvantages associated with the above-described conventional methods. Moreover, the operation time of these conventional methods usually take a time period of 2 to 9 hours, which not only is time-consumed, but also might affect the quality of the cell during separation. Therefore, these conventional methods do not have perfect design and need further improved.

In light of these disadvantages associated with those conventional cell separation methods, the inventor of this application devoted to improve, and finally, after extensive study for many years, has developed successfully the apparatus and method for rapid separation of cells according to the invention.

### SUMMARY OF THE INVENTION

The invention provides an apparatus and a method for rapid separation of cells without using density gradient and antibodies, characterized in that, in addition to provide simpler and rapid operation procedure, as well as need not use antibody, a more economic apparatus and method can be realized so as to lower the cost involved in research and development.

The invention provides an apparatus and a method for rapid separation of cells without using density gradient and antibodies, characterized in that it needs neither particular chemical agent for producing density gradient nor antibodies for recognizing antigen such that the quality and integrity of the cell separated can be assured.

The invention provides an apparatus and a method for rapid separation of cells without using density gradient and antibodies, characterized in that it can offer an apparatus and method for separating rapidly and efficiently sub-populations of mononuclear cells.

The invention provides an apparatus and a method for rapid separation of cells without using density gradient and antibodies, characterized in that it can be used in the screening of drugs so as to provide researchers a more convenient and effective tools for drug screening.

The invention provides a continuous separation apparatus to be able to use in the continuous analytical application of current flow cytometer and other analyzers.

The invention provides a column that can be used in the separation of cells, even the separation of sub-population of mononuclear cells, based on the principle of interaction between cell surface and ionic exchange resin. As shown in FIG. 1, since numerous drugs act on the surface receptor of a cell to modify further the physical properties of the cell surface, different kinds of cells will have its own different retention time in the column in the presence or absence of drug action. Therefore, a column developed based on this principle can be used in drug screening.

These features and advantages of the present invention will be fully understood and appreciated from the following detailed description of the accompanying Drawings.

### BRIEF DESCRIPTION OF THE DRAWINGS

FIG. 1 is a schematic view showing the operation and the principle of the invention;

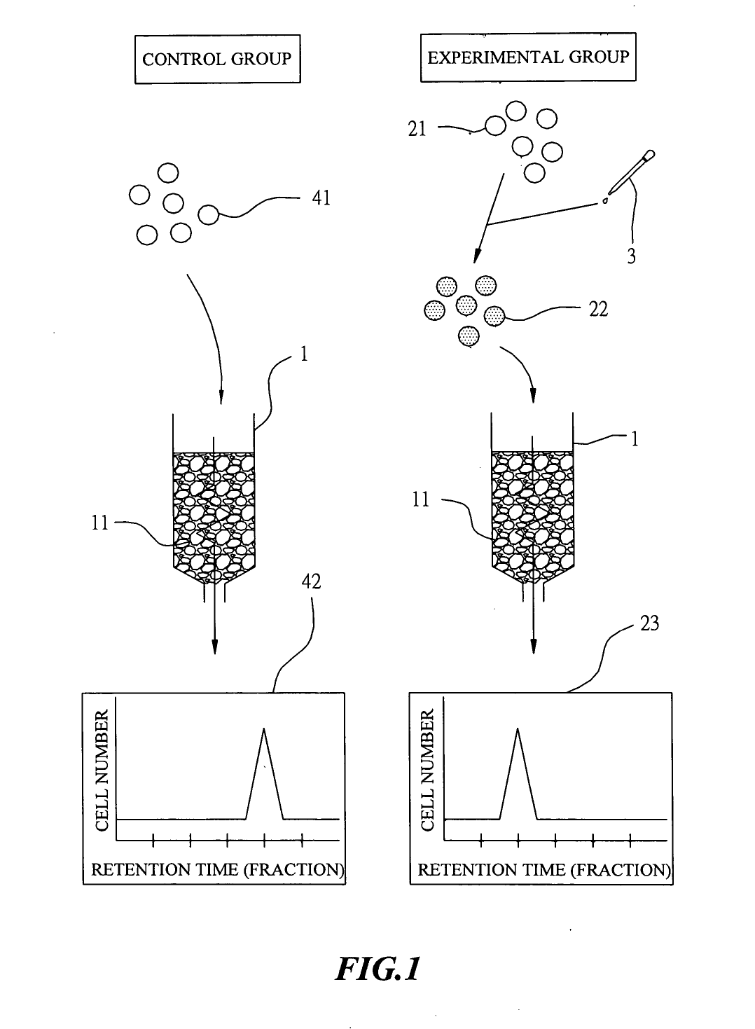

FIG. 2 is a chart showing the number of mononuclear cells collected at different time period during the separation of cells pre-treated with or without lectin on a small column;

FIG. 3 is a chart showing the number of erythrocytes collected at different time period during the separation of erythrocytes-containing mononuclear cells on a small column;

FIG. 4 is a chart showing the number of mononuclear cells collected at different time period during the separation of a concentrated suspension of mononuclear cells;

FIG. 5 is a chart showing the percentage of each sub-population of mononuclear cells collected in the separation on a small column;

FIG. 6 is a chart showing the number of mononuclear cells collected at different time period during the separation of a concentrated mononuclear cells suspension on a large column; and

FIG. 7 is a chart showing the percentage of each sub-population of mononuclear cells collected on a large column.

### DETAILED DESCRIPTION OF THE PREFERRED EMBODIMENT

The apparatus and method for rapid separation of cells without using density gradient and antibody provided according to the invention comprises a column for separating cells, wherein the column can be made of, for example, glass, plastics or metal, and is packed with resin particles having a size of 100 to 400 micrometer, and wherein the resin may be polystyrene or polyvinyl chloride (PVC), or resin modified with a chemical substance or specific chemical functional group such as, for example, —CN, propyl, phenyl, hydroxylapatite, long chain carbon, NH₃, N,N,N-trimethyl amine (N(CH₃)₃), N,N-diethylamine (N(C₂H₅)₂), or N,N-dimethylamine (N(CH₃)₂) that may be positively charged, and sulfite (SO₃⁻) or carboxyl group (COO⁻) that may be negatively charged. Those chemical substances can be a glycosyl substance such as, for example, a pyranyl, a furanyl, a polysaccharide, or amino acids constituting a protein. The column can take a shape of a cylinder and can be a capillary tube in any size of diameter and length varying as desired.

Cells to be separated by the apparatus and method for separating cells according to the invention can be blood cells, or a suspension of attached cells undergone dissociation.

The invention will be illustrated by following non-limiting examples.

### EXAMPLE 1

### Interaction of Cell and Chemical

In this example, the column used for separating cell has an inner diameter of 6 mm, a length of 180 mm and a volume of 5 ml. This column was packed with resin particles and was washed first and thereafter, filled with a phosphate buffered saline (PBS).

### The Control Group

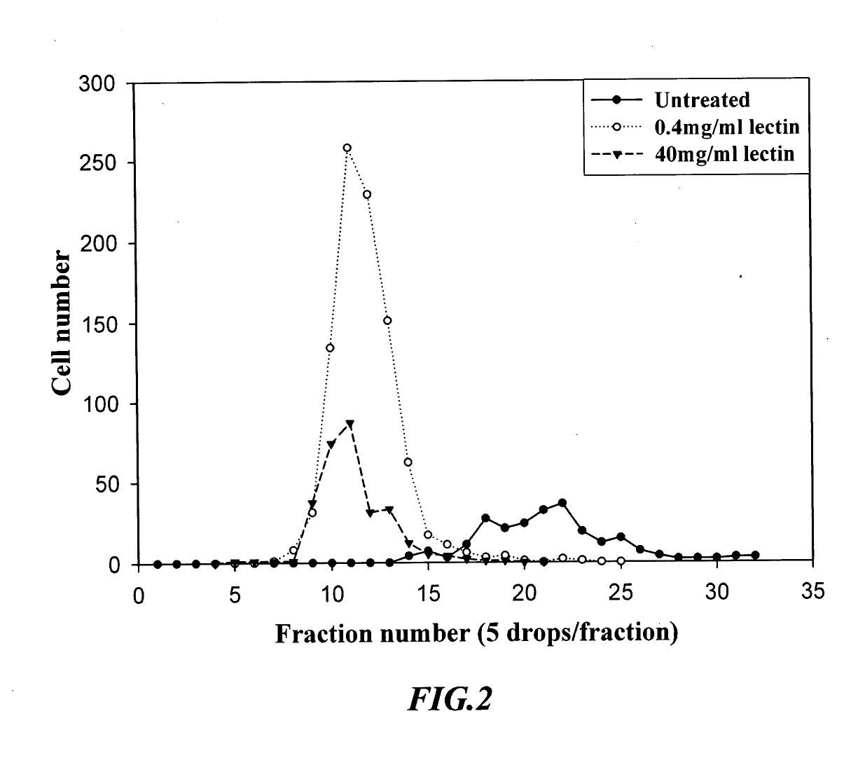

Blood sample was separated first by centrifugation using dextran cell separation medium (Ficoll) to yield peripheral blood mononuclear cell (PBMC). The thus-obtained PBMC was diluted with phosphate buffered saline (PBS) into a cell suspension at concentration of 1×10⁶ cells/ml. This cell suspension was loaded then on the upper frontier of the above-described column. After adding PBS to a level of a pre-determined height, the column was eluted with PBS at a flow rate of 3 ml/min and cell fractions were collected in test tubes, respectively, in a manner that each test tube collected 5 drops of cell suspension eluent. Thereafter, the eluted cell suspension in each test tube was examined under an optical microscope and counted by a cell counter. The result was showed in FIG. 2.

### Experimental Group

Blood sample was separated first by centrifugation using dextran cell separation medium (Ficoll) to yield peripheral blood mononuclear cell (PBMC). The thus-obtained PBMC was diluted with phosphate buffered saline (PBS) into a cell suspension at concentration of 1×10⁶ cells/ml. The cell suspension was added with lectin at a concentration of 0.4 μg/ml or 40 μg/ml. After reacting for 5 minutes, cells thus treated with lectin was loaded on the upper frontier of the above-described column, and after adding PBS to a level of a pre-determined height, the column was eluted with PBS at a flow rate of 3 ml/min and cell fractions were collected in test tubes, respectively, in a manner that each test tube collected 5 drops of cell suspension eluent. Thereafter, the eluted cell suspension in each test tube was examined under an optical microscope and counted by a cell counter. The result was showed in FIG. 2.

As shown in FIG. 2, cells treated with lectin (the experimental group) was eluted out of the column before non-treated cells (the control group), which demonstrated lectin interacted with cells and modified the surface molecules on the cell in a manner that the hydrophilic property of the cell surface was increased, while the interaction with polystyrene was decreased, such that the cell treated with lectin was eluted earlier. Consequently, this method can be used to evaluate and analyze the interaction of cells with chemical molecules, nucleic acids, and proteins.

### EXAMPLE 2

### Separation of Sub-Population of Mononuclear Cells

### EXAMPLE 2(A)

In example 2(A), the column used for separating cell has an inner diameter of 6 mm, a length of 180 mm and a volume of 5 ml. This column was packed with resin particles and was washed first and thereafter, filled with a phosphate buffered saline (PBS).

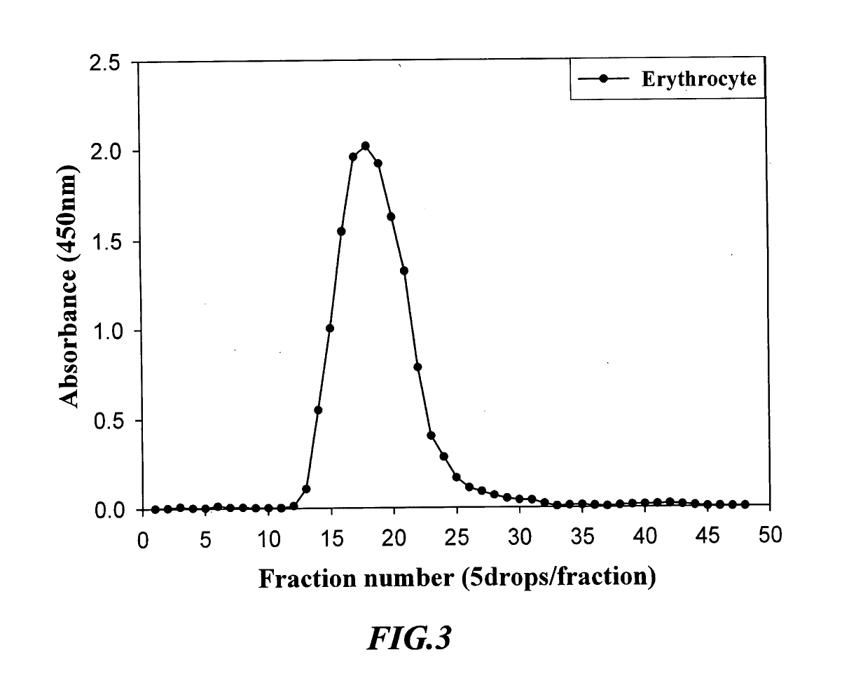

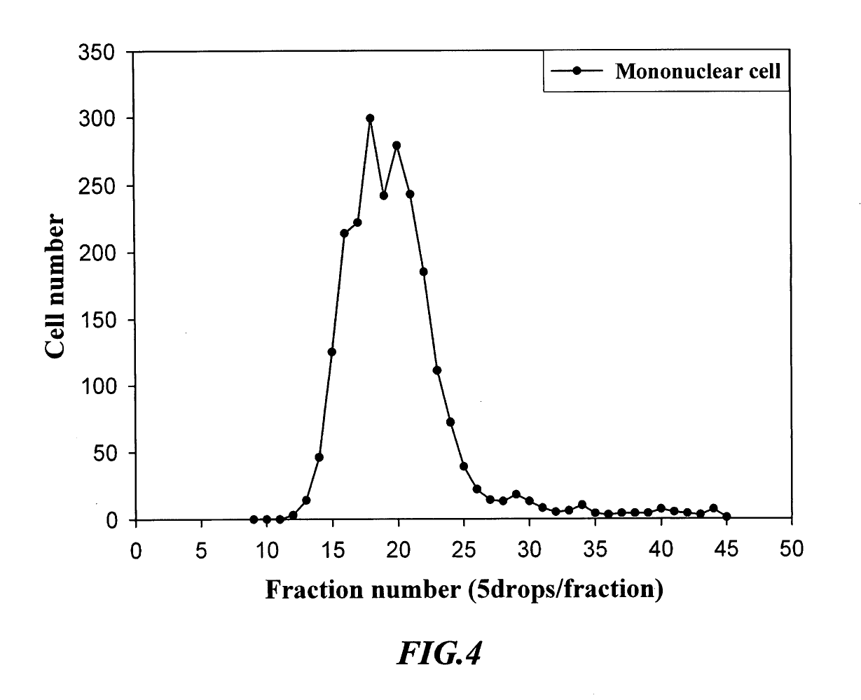

A concentrated mononuclear cells suspension was diluted with PBS to a cell suspension at a concentration of 1×10⁶ mononuclear cells/ml, containing still a certain amount of erythrocytes. This cell suspension was loaded then on the above-described column. The column was eluted subsequently with PBS at a flow rate of 3 ml/min and cell fractions were collected in test tubes, respectively, in a manner that each test tube collected 5 drops of cell suspension eluent. Thereafter, the eluted cell suspension in each test tube was examined under an optical microscope and numbers of erythrocytes and leukocytes were counted by a cell counter, respectively. The results were shown in FIG. 3 and 4. FIG. 3 shows the number of erythrocytes, while FIG. 4 shows the number of mononuclear cells.

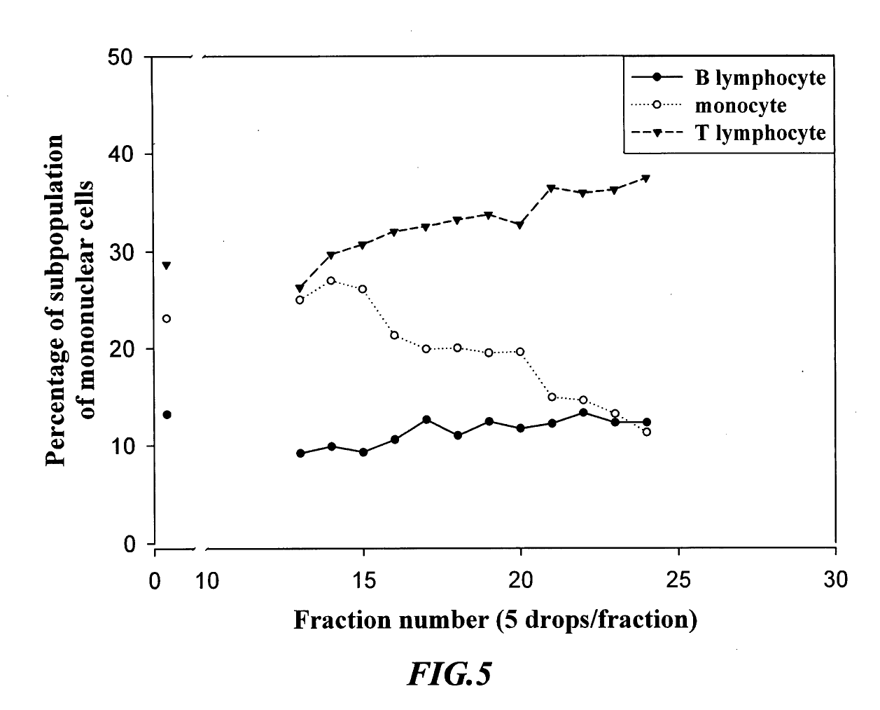

Since each sub-population of mononuclear cells has to be recognized with each own antibody, to the cell suspension eluent in each collecting tube was added 0.1 μg of anti-CD3-FITC, anti-CD19-PE and anti-CD14-Cy5 antibodies conjugated with fluorescent substances, in order to recognized T lymphocyte (CD3⁺), B lymphocyte (CD19⁺), and monocyte (CD14⁺), respectively. After carrying out a fluorescent immuno-staining analysis by a flow cytometer, the relative percentages of each sub-population were shown in FIG. 5.

### EXAMPLE 2(B)

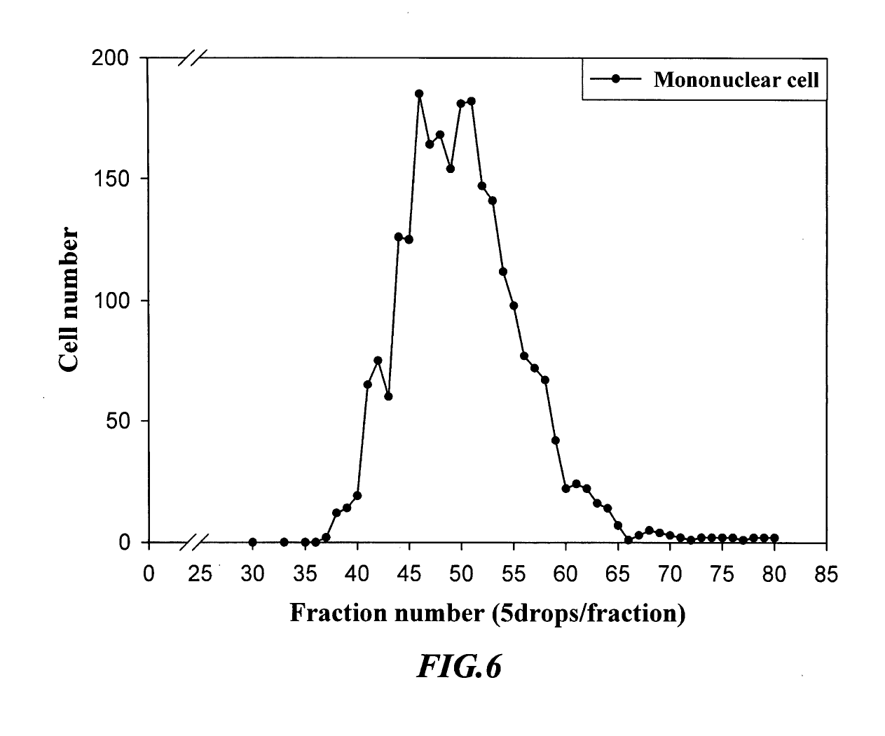

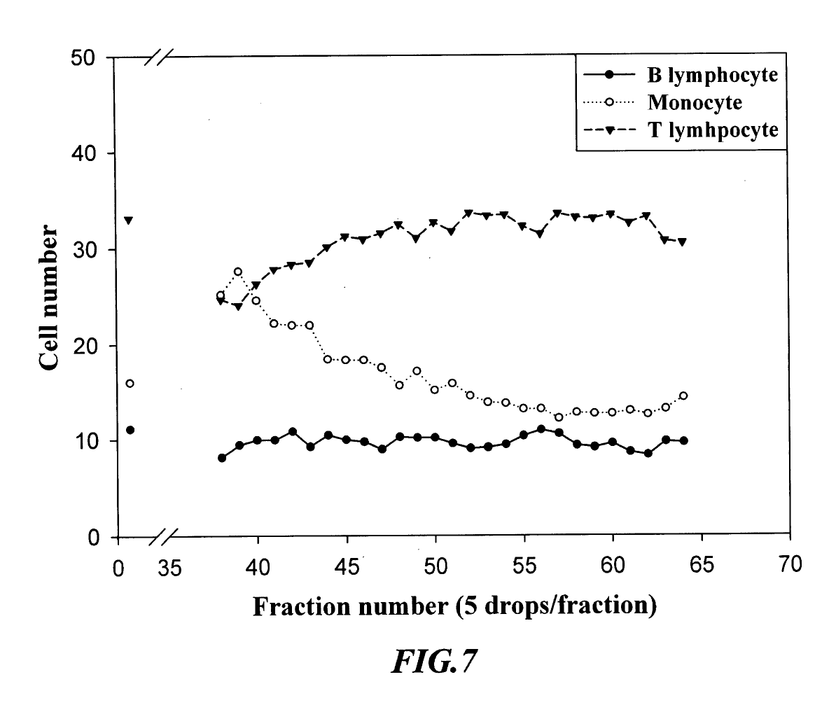

In example 2(B), the column used for separating cell was changed and has an inner diameter of 8 mm, a length of 200 mm and a volume of 10 ml. The procedure of example 2(A) was repeated, and the column was filled at a flow rate of 1.2 ml/min. The result was shown in FIG. 6 and 7. FIG. 6 shows the number of mononuclear cells, while FIG. 7 shows the relative percentage of each sub-population of mononuclear cells.

The results obtained above suggest that the column could not achieve any separation effect against a single population of erythrocyte as shown in FIG. 3. For mononuclear cells in a same sample, several bands were eluted successively, as shown in FIG. 4 and 6. After analyzing further by fluorescence immuno-staining, the column provided a partition effect with respect to various sub-populations of mononuclear cells such as, T lymphocyte, B lymphocyte and monocyte, and revealed a significantly difference variation, as shown in FIG. 5 and 7.

The size of the inner diameter and length of the column might have a slight influence on the separation effect. The less the diameter of the column is, the better separation effect can be obtained, as demonstrated in FIG. 5 and 7. For the small column, as shown in FIG. 5, after separating on a small column, T lymphocytes had its percentage increased from 26% (the 13^{th} collection tube) to 39% (the 24^{th} collection tube), which corresponding to a increase of 50% over the original sample, while monocytes had its percentage decreased from 25% (the 13^{th} collection tube) to 10% (the 24^{th} collection tube), which corresponding to a decrease of about 60% over the original sample. Therefore, with this method, by collecting cells eluted at different time period, each sub-population of mononuclear cells can be rapidly and efficiently separated. Obviously, this method can be applied not only for the general separation of blood cells, but also for clinical separation and removal of cancer cells from leukemia patient.

The apparatus and method for rapid separation of cells without using density gradient and antibody provided according to the invention has several following advantages over prior patent and conventional techniques recited above:
1. The apparatus and method according to the invention has a simpler and rapid operation procedure to achieve the effect of cell separation without using any antibody, and hence provides a more economical apparatus and method to reduce the cost of research and development.
2. The apparatus and method according to the invention can achieve the desired cell separation without using any special chemicals such that the quality of cells thus separated can be assured.
3. The apparatus and method according to the invention can separate rapidly and efficiently each sub-population of mononuclear cells without using any antibody, thereby the cell thus separated can be used directly for further research and development as well as for clinical application.
4. The apparatus and method according to the invention can be used for evaluating and analyzing the interaction of cells with chemical molecules, nucleic acids, and protein, and is applicable further in the screening of drugs.
5. The apparatus and method according to the invention can be used in continuous separation process, and can be used in combination with other analytical instruments in a continuous and timely analytical system.
6. The apparatus and method according to the invention can be applied in the usual separation of blood cells for research use, as well as for the separation and removal of cancer cells from the clinical leukemia patients.

While the above description gives only a specific illustration for an embodiment of the invention, it is understood that the embodiment is not used to limit the scope of the invention. Any equivalent variation and modification not departing from the spirit of the invention, such as, equivalent embodiments on the size of the inner diameter and length of the column, as well as variation on the packed resin particles, are considered to be fallen within the scope of the appended claims.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. An apparatus for rapid separation of cells without using density gradient and antibody, comprising a column and packing material in the column, wherein the column is used for separating different populations of the cells.

2. An apparatus for rapid separation of cells without using density gradient and antibody as recited in claim 1, wherein the column is made of material such as, glass, plastics or metal.

3. An apparatus for rapid separation of cells without using density gradient and antibody as recited in claim 1, wherein the column can take a shape of cylinder, or a capillary tube, and therein the column can have an inner diameter and a length varied as predetermined.

4. An apparatus for rapid separation of cells without using density gradient and antibody as recited in claim 1, wherein the packing material in the column is a resin, polystyrene or polyvinyl chloride (PVC).

5. An apparatus for rapid separation of cells without using density gradient and antibody as recited in claim 4, wherein the packing material can be modified further with a chemical substance or a specific chemical functional group.

6. An apparatus for rapid separation of cells without using density gradient and antibody as recited in claim 5, wherein the specific chemical functional groups is —CN, propyl, phenyl, hydroxylapatite, long chain carbon, NH₃, N,N,N-trimethyl amine (N(CH₃)₃), N,N-diethylamine (N(C₂H₅)₂), or N,N-dimethylamine (N(CH₃)₂) that may be positively charged, and sulfite (SO₃⁻) or carboxyl group (COO⁻) that may be negatively charged.

7. An apparatus for rapid separation of cells without using density gradient and antibody as recited in claim 5, wherein the chemical substance is a glycosyl substance or an amino acid.

8. An apparatus for rapid separation of cells without using density gradient and antibody as recited in claim 7, wherein the glycosyl substances is a pyranyl substance or a furanyl substance as well as a polysaccharide.

9. An apparatus for rapid separation of cells without using density gradient and antibody as recited in claim 7, wherein the amino acids is one of the 20 amino acids constituting a protein.

10. An apparatus for rapid separation of cells without using density gradient and antibody as recited in claim 1, wherein the cells are blood cells or cell suspensions of attached cells undergone dissociation.

11. A method using an apparatus with a column and packing material in the column, wherein the column is used for separating different populations of cells, for rapid separation of the cells without using density gradient and antibody, wherein a cell sample is diluted with a buffer solution to a cell suspension having a pre-determined concentration, the cell suspension is loaded on the apparatus and is eluted with a buffer solution at a constant flow rate, the cell suspension eluent is collected in a test tube in a manner that a collecting test tube is successively replaced at a time interval or after collecting a constant number of cell suspension drop, and finally, the collected cells is identified into respective cell population so as to analyze the quality of cells thus separated.

12. A method as recited in claim 11, wherein the constant flow rate is a flow rate achieved under naturally dropping or a steady flow rate provided under the action of a pump.

13. A method as recited in claim 11, wherein the loading of the sample is carried out manually or automatically by a sampling machine.

14. A method as recited in claim 11, wherein the identification of cell populations after separation is carried out by integrating with a cell counter to count the cell number, or by immuno-fluorescence staining analysis using a combined flow cytometer.
